(19) United States
(12) Patent Application Publication
(10) Pub. No.: US 2012/0064478 A1
(43) Pub. Date: Mar. 15, 2012

(54) METHOD AND ACCESSORY FOR USE DURING DENTAL PROCEDURE INCLUDING MIRROR FOR PATIENT

(76) Inventor: Nicole Coriat, Thornhill (CA)

(21) Appl. No.: 13/232,724

(22) Filed: Sep. 14, 2011

Related U.S. Application Data
(60) Provisional application No. 61/382,689, filed on Sep. 14, 2010.

(51) Int. Cl. A61B 1/247; A61C 17/00; A61C 5/04

(52) U.S. Cl. 433/30; 433/215; 433/226; 433/216

(57) ABSTRACT

In a first aspect, the invention is directed to an accessory for use by a dental professional. The accessory includes a mirror and a connector. The connector has a connector body having a first end and a second end. The connector includes an eyewear mount at the first end and a mirror mount at the second end. The eyewear mount is configured to mount to eyewear. The connector is adjustable to position and hold the mirror in a plurality of orientations in which the mirror faces generally forwardly of the dental professional.

### FIELD OF THE INVENTION

The present invention relates to accessories for use during dental procedures.

### BACKGROUND OF THE INVENTION

Dental professionals, such as dental hygienists and dentists, when providing a service to a patient, such as, for example, a dental cleaning, or an examination, often find points of interest in the patient's mouth, which they discuss with the patient. Such points of interest may be, for example, areas to which the patient should pay particular attention when brushing their teeth, cavities that have formed, fillings that require replacement, gum recession, and/or other dental issues that can arise.

While such discussions are useful, the patient is sometimes left with little incentive to improve their oral hygiene.

### SUMMARY OF THE INVENTION

In a first aspect, the invention is directed to an accessory for use by a dental professional. The accessory includes a mirror and a connector. The connector has a connector body having a first end and a second end. The connector includes an eyewear mount at the first end and a mirror mount at the second end. The eyewear mount is configured to mount to eyewear. The connector is adjustable to position and hold the mirror in a plurality of orientations in which the mirror faces generally forwardly of the dental professional.

In a second aspect, the invention is directed to a method for conducting a dental procedure on a patient, comprising:

providing eyewear with a mirror thereon;

conducting dental work on the patient; and

orienting the mirror forwardly at a position selected so that the patient can see a point of interest in the mouth of the patient. The dental work may include at least one action selected from the group of actions consisting of: filling a cavity; replacing a filling; carrying out a cleaning; carrying out dental surgery; and examining the state of oral health of the patient.

### BRIEF DESCRIPTION OF THE DRAWINGS

The present invention will now be described by way of example only with reference to the attached drawings, in which:

FIG. 1 is a perspective view of eyewear with an accessory attached thereto in accordance with an embodiment of the present invention, the accessory including a mirror and a connector;

FIG. 2 is a side view of a portion of the connector shown in FIG. 1;

FIG. 3 is a side view of a portion of the connector shown in FIG. 1; and

FIG. 4 is an illustration of a dental professional using the accessory to show a patient the patient's teeth.

### DETAILED DESCRIPTION OF THE INVENTION

Reference is made to FIG. 1, which shows an accessory 10 for use by a dental professional, in accordance with an embodiment of the present invention. The accessory 10 includes a mirror 12 and a connector 14. The connector 14 includes a connector body 15, an eyewear mount 16 and a mirror mount 17.

The connector body 15 may have any suitable structure. For example, the connector body 15 may be made up of a metallic core member 18 that is flexible and that remains in whatever position it is moved to, and a polymeric cover layer 19 that is sufficiently flexible to conform to whatever shape the core member 18 takes on. The connector body 15 preferably is made in such a way to be easily cleanable after use so as to inhibit the growth of bacteria thereon that can inadvertently be transferred to a patient.

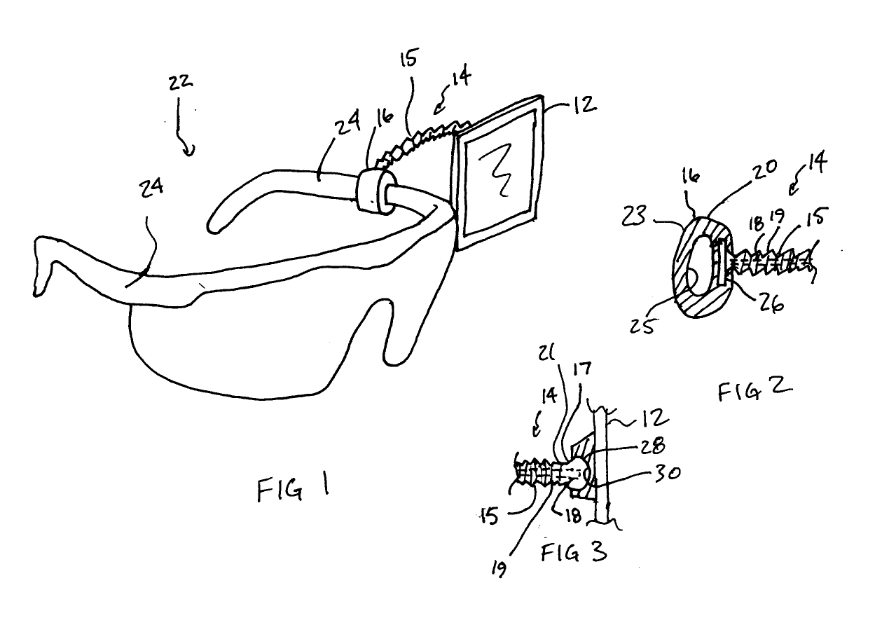

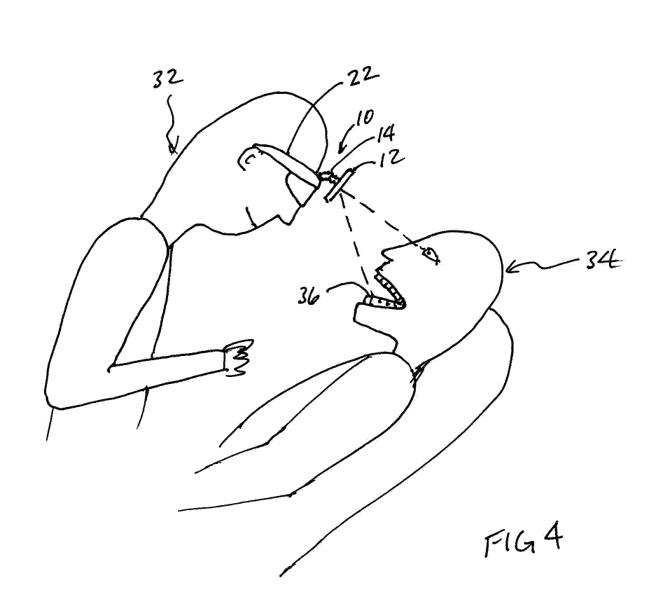

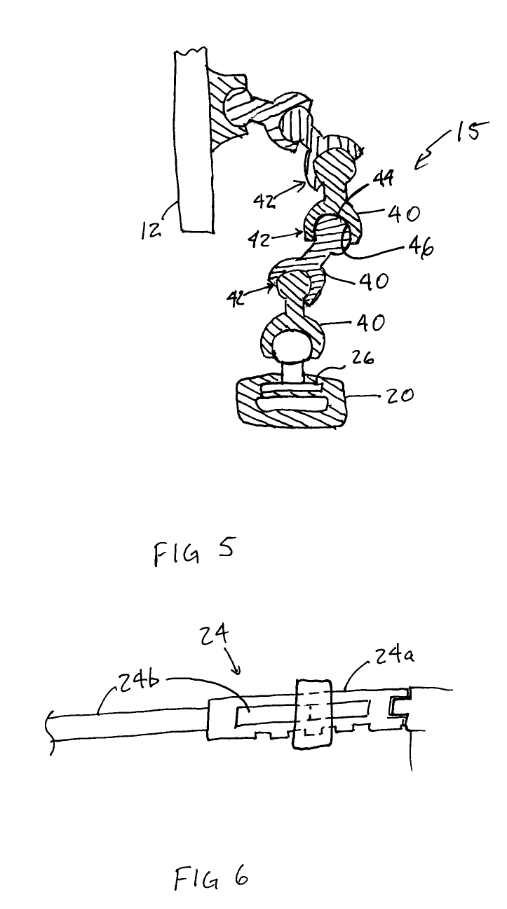

The connector body 15 has a first end 20 and a second end 21 (FIG. 3). Referring to FIG. 2, the eyewear mount 16 is provided at the first end 20 and is configured to mount to eyewear, shown at 22. The eyewear 22 in the exemplary embodiment shown in FIG. 1 is protective eyewear such as is worn by a dental hygienist while carrying out a dental cleaning on a patient. It is alternatively possible for the eyewear 22 to be any suitable eyewear, however, such as prescription eyeglasses.

The eyewear mount 16 itself may comprise a resilient ring or sleeve 23 of material having an inner surface 25 that is grippy (i.e. that has a selected coefficient of friction), such as, for example, rubber. The ring 23 is sized to fit over the arm of the eyewear 20, shown at 24, and to be moved to a selected point on the arm 24. The ring 23 may be sufficiently resilient and expandable to fit over the arms of different types of eyewear. Furthermore, the resiliency of the ring 23 preferably permits the ring 23 to be adjusted longitudinally along the arm 24 of the eyewear. In situations where the eyewear has arms that are adjustable, as shown in FIG. 6, it will be noted that the inner surface 25 of the ring is sufficiently smooth and has sufficiently low friction at least in selected areas to permit relative movement to take place between a front portion 24a of the arm 24 and a rear portion 24b of the arm 24. A plate, shown at 26 in FIG. 2, may be molded into the ring 22 to provide stability for supporting the weight of the mirror 12. The plate 26 may be part of the connector body 15. When the accessory 10 is mounted to the arm 24 of the eyewear 22, the material at the inner surface 25 of the ring 23 is preferably selected to grip the arm 24 with sufficiently high friction that it prevents the ring 23 from rotating or otherwise moving about the arm 24 under the weight of the mirror 12 being supported. When it is desired to clean the eyewear 22 and the accessory 10, the ring 23 is preferably capable of being removed from the arm of the eyewear 22.

The mirror mount 17 is at the second end 21 of the connector body 15, may have any suitable structure. For example, the mirror mount 17 may be a ball 28 (FIG. 3) that is captured in a socket 30 on the mirror 12 to form a ball joint.

As an alternative to the structure shown in FIG. 2, the connector body 15 may have another structure. For example, with reference to FIG. 5, the connector body 15 may be formed by a plurality of polymeric links shown at 40, wherein each link 40 connects to each subsequent link 40 by a ball joint 42 that is sufficiently tight-fitting that the links 40 remain in whatever position they are moved to as a result of friction at mating ball and socket surfaces shown at 44 and 46 in the joints 42. The ball joint may be similar to the ball joint formed between the connector 14 and the mirror 12 shown in FIG. 3. The ball joint is just one type of multi-axis joint that is possibly between links 40. Other types of multi-axis joint are possible. For example, it is possible to provide joints that include both a swivel function and a hinge function. A swivel function would provide movement in one dimension (a rotational dimension) and the hinge function would provide movement in a second dimension, (also a rotational dimension). It is alternatively possible to provide joints between adjacent links 40 whereby each joint only has movement in one dimension, but in total the joints provide movement in three dimensions.

As a result of the flexibility of the connector body 15, the connector 14 is adjustable to position and hold the mirror 12 in a plurality of orientations in which the mirror 12 faces generally forwardly of the dental professional 32. As shown, the connector 14 is adjustable in three independent dimensions so as to permit the mirror 12 to be positioned to reflect the patient's mouth to the patient's eyes at different positions and orientations of the dental professional's head relative to the patient's head. For example, if the dental professional 32 is carrying out an action or an inspection on the patient's upper rear teeth, the professional 32 may have their head positioned and oriented in a certain position and orientation, whereas when the professional is carrying out an action or an inspection on the patient's lower front teeth, the professional may have their head positioned and oriented in a different position.

The mirror 12 may have any suitable shape, such as, for example, circular, rectangular, square, or a flower-shape. The mirror 12 is shown with a peripheral frame, however, it is optionally possible for the mirror 12 to be frameless so as to avoid the presence of seams or crevices which can trap blood or other bio-products and which can be difficult to clean. In one embodiment, the dimensions of the mirror 12 may be approximately 4.5 cm to approximately 5 cm in width and in height.

The rear of the mirror 12 has the socket 30 thereon for receiving the ball 28. Preferably, the socket 30 is configured to permit the removal of the ball 28 so that their mating surfaces can be cleaned after use.

Use of the accessory 10 is illustrated in FIG. 4. During use, the dental professional, shown at 32, can adjust the mirror 12 so that the mirror 12 is positioned to permit the patient, shown at 34, to view inside their mouth while the professional 32 is carrying out a procedure. This can be particularly useful in situations when the professional 32 wants to show the patient 34 a particular point of interest, shown at 36, in the patient's mouth. Furthermore, in some situations both hands of the professional 32 are occupied and so it is not possible for the professional 32 to hold a mirror. Furthermore, in some situations, the patient 34 can view substantially the entire procedure that is being carried out. In each of these situations, the patient 34 can see the points of interest 36 in their mouth rather than simply hearing about them from the professional 32. Thus, when the dental professional 32 explains where in the patient's mouth there has been, for example, excessive tartar build-up, or for example, poor gum health, the professional 32 can show the patient 34 exactly where in the patient's mouth these problems exist. Thus, when the professional 32 urges the patient 34 to brush more thoroughly in those areas, the patient 34 has visual feedback on what damage has resulted from their poor hygiene. Additionally, the patient 34 has visual feedback on where exactly the issues are. By receiving the visual feedback in addition to the verbal feedback provided by the professional 32, the patient 34 may be left with a stronger impression of what damage is present in their mouth (as compared with receiving verbal feedback only), and the patient 34 may thus be more inclined to follow through on the urgings of the professional 32 to improve their oral hygiene.

The aforementioned use of the mirror 12 is particularly advantageous during a dental cleaning.

In a preferred embodiment, the connector body 15 is sufficiently flexible that, at times or in situations where the patient 34 does not wish to see their mouth, the mirror 12 may be positioned out of the way, eg. parallel to and adjacent the side of the head of the professional 32.

In a preferred embodiment, the materials used for the cover layer 19 of the connector 14, and for the frame of the mirror 12 (if provided), are selected to create a positive atmosphere for the patient, and as such, they may be attractively coloured, using any suitable colour. The mirror frame may be differently coloured than the cover layer 19 of the connector.

The dental procedure, which may also be referred to as dental work, may include at least one action selected from the group of actions consisting of: filling a cavity; replacing a filling; carrying out a cleaning; carrying out dental surgery; and examining the state of oral health of the patient. The dental work may alternatively or additionally include any other type of work carried out by dental professionals, such as dentists, dental surgeons, dental hygienists and the like on a patient's mouth.

While the above description constitutes a plurality of embodiments of the present invention, it will be appreciated that the present invention is susceptible to further modification and change without departing from the fair meaning of the accompanying claims.

1. An accessory for use by a dental professional during a dental procedure by the dental professional on a patient, comprising:
a mirror; and
a connector having a connector body having a first end and a second end, wherein the connector includes an eyewear mount at the first end and a mirror mount at the second end, wherein the eyewear mount is configured to mount to eyewear, wherein the connector is adjustable to position and hold the mirror in a plurality of orientations in which the mirror faces generally forwardly of the dental professional, so as to permit the patient to see a point of interest in the mouth of the patient.

2. An accessory as claimed in claim 1, wherein the eyewear mount includes a sleeve that has an interior surface that defines an arm pass-through aperture, wherein the sleeve is resiliently expandable to accommodate different sizes of arms of eyewear, and has an interior surface that includes a grip surface that has a selected coefficient of friction so as to grip a portion of the arm of the eyewear, wherein the sleeve is removable from the arm of the eyewear.

3. An accessory as claimed in claim 1, wherein the eyewear mount includes a sleeve that has an interior surface that defines an arm pass-through aperture, wherein the sleeve is resiliently expandable to accommodate different sizes of arms of eyewear, wherein the interior surface includes a grip surface that has a selected coefficient of friction so as to grip a portion of the arm of the eyewear, wherein the sleeve is sufficiently resiliently expandable to permit deformation of the sleeve so as to permit adjustment in position of the sleeve longitudinally along the arm of the eyewear.

4. An accessory as claimed in claim 1, wherein the eyewear mount includes a sleeve that has an interior surface that defines an arm pass-through aperture, wherein the sleeve is resiliently expandable to accommodate different sizes of arms of eyewear, wherein the interior surface accommodates relative sliding motion between a rear portion of the arm and a front portion of the arm.

5. An accessory as claimed in claim 1, wherein the connector is adjustable in position in three independent dimensions so as to permit the adjustment of the mirror relative to the eyes and mouth of the patient over a range of positions of the dental professional.

6. An accessory as claimed in claim 1, wherein the connector includes a plurality of connector elements that are connected to one another via multi-axis joints and are holdable in position relative to one another via friction in the multi-axis joints.

7. A method for conducting a dental procedure on a patient, comprising:
donning eyewear with a mirror thereon;
conducting dental work on the patient; and
orienting the mirror forwardly at a position selected so that the patient can see a point of interest in the mouth of the patient.

8. A method as claimed in claim 7, wherein the dental work includes at least one action selected from the group of actions consisting of: filling a cavity; replacing a filling; carrying out a cleaning; carrying out dental surgery; and examining the state of oral health of the patient.

9. A method as claimed in claim 7, wherein the orienting step includes orienting the mirror in three independent dimensions.
